(19) United States
(12) Patent Application Publication
(10) Pub. No.: US 2015/0133643 A1
(43) Pub. Date: May 14, 2015

(54) Low Organic Extractable Depth Filter Media Processed with Solvent Extraction Method

(71) Applicant: EMD Millipore Corporation, Billerica, MA (US)

(72) Inventors: Kwok-Shun Cheng, Nashua, NH (US); Nripen Singh, Acton, MA (US)

(21) Appl. No.: 14/400,392

(22) PCT Filed: Jun. 6, 2013
(86) PCT No.: PCT/US2013/044550

§ 371 Date: Nov. 11, 2014

Related U.S. Application Data
(60) Provisional application No. 61/656,263, filed on Jun. 6, 2012.
(60) Provisional application No. 61/664,999, filed on Jun. 27, 2012.

(51) Int. Cl. C07K 1/34; C07K 16/00

(52) U.S. Cl. CPC C07K 1/34; C07K 16/00
USPC 530/388.1; 530/387.1

(57) ABSTRACT

Provided is a primary clarification depth filtration process of cell-culture feeds, including chemically treated flocculated feeds, containing target biomolecules of interest such as mAbs, mammalian cell cultures, or bacterial cell cultures, utilizing a primary clarification depth filtration device containing a media with significantly lower flushing requirements, resulting in lower levels of organic extractables released after media flushing, and increased throughput for the pre-treated feed streams, without the use of a primary clarification centrifugation step or primary clarification tangential flow microfiltration step. The primary clarification depth filtration device used in the primary clarification of fluid cell culture feeds, including chemically treated flocculated feeds containing flocculated cellular debris and/or colloidal particulates having a particle size distribution of about 0.5 μm to 200 um, contains a porous depth filter media having porous layers of varying pore ratings, and achieves the desired level, of total organic extractables (1-3 ppm) measured in the feed filtered through the media with, significantly lower flushing requirements. Kits and methods of using and making the same are also provided.

### RELATED APPLICATIONS

The present patent application claims the benefit of priority of U.S. provisional patent application No. 61/656,263, filed Jun. 6, 2012 and U.S. provisional patent application No. 61/664,999, filed Jun. 27, 2012, the entire contents of which are incorporated by reference in their entirety.

### DESCRIPTION OF THE INVENTION

1. Field of the Invention

In general, the present invention relates to lower organic extractable media used in the primary clarification of cell culture feeds. In certain specific embodiments, the invention provides a primary clarification depth filtration process of cell-culture feeds and the like, which utilizes a primary clarification depth filtration device containing a porous media with significantly lower flushing requirements resulting in lower levels of organic extractables released from the media after flushing, as well as having an increased throughput for the pre-treated feed streams, without the use of a primary clarification centrifugation step or primary clarification tangential flow microfiltration step.

2. Background of the Invention

Manufacturing pharmaceutical-grade biomolecules, including proteins such as monoclonal antibodies (mAbs), is a complex manufacturing process comprised of multiple filtration, centrifugation, and chromatography techniques designed to produce high quality products for patients. The clarification of cell culture harvests and high-solids feedstocks can be a daunting task due to the large volumes of harvest from modern production batch bioreactors (≦25,000 L) and high cell densities that often require primary, as well as secondary clarification prior to the subsequent chromatography operations. And as such, harvest and clarification schemes for the production processes of cell culture harvests and high-solids feedstocks, such as mammalian cells and mAbs, are the product of much evolution and evaluation carried out over the last 20 years or so.

Harvest techniques for mammalian cell culture and mAbs are now routinely expected to operate with high yields (>95%) and minimal cell disruption. As product molecule titers have increased, the higher cell mass and larger amounts of product create challenges for the downstream purification steps. Higher cell densities result in difficulties during clarification and sterile filtration. Higher product concentrations generally result in increased impurity load and the need for larger chromatography installations. As such, improvements in the form of gains in efficiency and throughput are greatly sought after.

Primary clarification of feeds, feedstreams, feedstocks, cell culture broths and the like, including high solids feeds, such as those containing mAbs and mammalian cell culture feedstocks, remove large amounts of biomass, particularly whole cells and other larger cellular debris, followed by secondary clarification which removes smaller colloidal particulates and other particles that impair the capacity of downstream filters. Centrifugation is typically the primary clarification step in the production processes of mAbs and mammalian cell culture broths and feedstocks.

mAb manufacturers have invested a great deal of time and effort increasing the product titer of a feedstock. However, while higher titers increase cell culture productivity, it also produces feedstocks with larger amounts of biomass and cell debris content. Feeds containing such larger amounts of biomass and cell debris can produce high turbidity centrates after centrifugation. High turbidity centrates often reduce the throughput of the secondary clarification depth filter and the subsequent sterile filter used downstream of the centrifuge. The reduced throughput causes a range of problems from increased process cost to deviations in process procedures due to plugging of filters and long processing delays. Finally, the need for primary clarification using a centrifuge requires extensive, validated cleaning procedures between runs to attempt to reduce the risk of cross contamination between batches and therapeutic molecular species.

This is particularly problematic at pilot or clinical scale biotherapeutic production where it is desirable to process multiple products in a relatively short time. The centrifuge cleaning procedures slow down the pilot plant's ability to change over to the production of a different biomolecule, and greatly increase the risk of cross contamination between production runs. In addition, centrifugation cannot efficiently remove all particulates and cellular debris from these feedstocks in the primary clarification step, hence the need for the secondary clarification step utilizing depth filtration after the centrifugation step, but prior to the subsequent chromatographic steps.

Alternatively, successive filtration runs have proven useful in removing different-sized cell and cellular debris from feedstocks, but typically the volumetric throughputs limit the application to smaller volumes (<1000 L) where the filter installation has a reasonable size. The use of filtration greatly reduces the risk of cross contamination and eliminates the need for cleaning and cleaning validation between runs due the disposable nature of filtration devices. Unfortunately, the low throughput requires a large number of filter units which can reduce filtration yields because each successive step results in the loss of a portion of the feed solution through hold-up volumes of the filter device and equipment.

In order to further enhance clarification performance, throughput and downstream filtration operations, the flocculation of a cell culture harvests have been used. Flocculants precipitate cells, cell debris and proteins because of the interaction between the charges on the proteins and charges on the polymer (e.g. polyelectrolytes), creating insoluble complexes, and subsequent bridging of insoluble complexes either by residual charge interaction or through hydrophobic patches on the complexes to form larger clusters. In order to remove these large clusters, a centrifuging step or tangential flow microfiltration is the primary mode of clarification followed by the secondary clarification step whereby depth filtration is widely used in the clarification of cell culture broth prior to the capture chromatography step. Since centrifugation cannot deliver a particle-free centrate, depth filter (secondary depth filtration) and sterile filter need to be installed further downstream.

Tangential flow microfiltration (also called cross-flow microfiltration) competes with centrifugation for the harvest and clarification of mAbs and therapeutic products from mammalian cell culture. One advantage this technique offers is the creation of a particle-free harvest stream that requires minimal additional filtration. However, tangential flow microfiltration membranes used for cell culture harvests are often plagued with the problem of membrane fouling (i.e., irrecoverable declines in membrane flux), and typically require strict complex operating conditions, followed by a thorough cleaning regimen (as is also the case with a centrifuge) for each membrane after each use. One way to address the tangential flow microfiltration membrane fouling issue is by using more hydrophilic membranes, which are generally considered somewhat less susceptible to significant fouling.

Depth filter clarification media are extensively used to clarify cell-culture feeds and have demonstrated the ability to reduce turbidity and remove some soluble impurities such as DNA, host cell protein, and endotoxin. Depth filter clarification media are typically constructed from materials of a fibrous bed of cellulose, a wet-strength resin binder and an inorganic filter aid such as diatomaceous earth. The resin binder helps to impart wet tensile strength, provide an adsorptive charge to bind impurities and minimize shedding of materials of composition (i.e. cellulose and filter aid). The diatomaceous earth provides a high surface area to the filter and contributes to the adsorptive properties. However, two of the components (cellulose and resin binder) are known to contribute to organic extractables. Therefore, these depth filters need to be flushed prior to use to reduce the organic extractables which can be expensive and time-consuming. Based on the above mentioned limitations of existing filters, there is a need to develop next generation depth filter media with significantly lower flushing requirements and increased throughput for the pre-treated feed streams.

### SUMMARY OF THE INVENTION

In response to the above needs and problems associated with the primary clarification processes of feeds, feed streams, feedstocks, cell culture broths and the like, the present invention overcomes the challenges by using a primary clarification depth filtration process which utilizes a primary clarification depth filtration device containing a porous media with significantly lower flushing requirements resulting in lower levels of organic extractables released from the media after flushing, as well as having an increased throughput for the pre-treated feed streams.

The present invention also encompasses a process for reducing organic extractables released from a primary clarification depth filtration media such that the level of total organic extractables measured in a feed filtered through the porous media after flushing is about 1-3 ppm, the process comprising:
a) providing a depth filtration device having a porous depth filter media;
b) extracting from the media with organic solvents; and
c) flushing the media at flow rates ranging from about 10 litres/m²/hr to about 600 litres/m²/hr such that the level of total organic extractables measured in a feed stock filtered through the media after flushing is about 1-3 ppm.

The present invention also encompasses a primary clarification depth filtration process using a primary clarification depth filtration device containing a porous media with significantly lower flushing requirements resulting in lower levels of organic extractables released from the media after flushing:
a) providing a primary clarification depth filtration device having a porous depth filter media;
b) extracting from the media with organic solvents;
c) flushing the media at flow rates ranging from about 10 litres/m²/hr to about 600 litres/m²/hr such that the level of total organic extractables measured in a feed stock filtered through the media after flushing is about 1-3 ppm; and
d) running a feed stock through the media after flushing.

The present invention also encompasses a process for the primary clarification of feeds, feedstreams, feedstocks, cell culture broths and the like, containing a target biomolecule of interest and a plurality of cellular debris and colloidal particulates without the use of a primary clarification centrifugation step or a primary clarification tangential flow microfiltration step using a primary clarification depth filtration containing a porous media with significantly lower flushing requirements resulting in lower levels of organic extractables released from the media after flushing, the process comprising:
a) providing a primary clarification depth filtration device having a porous media with significantly lower flushing requirements resulting in lower levels of organic extractables released from the media after flushing;
b) providing a feed stream containing a target biomolecule of interest and a plurality of cellular debris and particulates, wherein the cellular debris and particulates have a particle size distribution of about 0.5 μm to about 200 μm;
c) contacting the porous depth filter media with the teed stream, such that the depth filter media is capable of filtering cellular debris and particulates having a particle size distribution of about 0.5 μm to about 200 μm at a flow rate of about 10 litres/m²/hr to about 300 liters/m² hr such that the level of total organic extractables measured in the feedstream filtered through the media after flushing is about 1-3 ppm; and
d) separating the target biomolecule of interest from the cellular debris and particulates without the use of a primary clarification centrifugation step or a primary clarification tangential flow microfiltration step.

The present invention further encompasses a process for the primary clarification of a flocculated feed containing a target biomolecule or biotherapeutic of interest and flocculated cellular debris, materials, and colloidal particulates using a primary clarification depth filtration device without the use of a primary clarification centrifugation step or a primary clarification tangential flow microfiltration step, the process comprising:
a) providing a depth filtration device containing a porous media with significantly lower flushing requirements resulting in lower levels of organic extractables released from the media after flushing;
b) providing a chemical flocculant;
c) providing a feed containing a target biomolecule of interest and a plurality of cellular material, debris and colloidal particulates;
d) combining the chemical flocculant to the feed;
e) forming chemically flocculated cellular materials, debris and colloidal particulates in the feed, and optionally chemically flocculating the target biomolecule of interest;
f) contacting the porous depth filter media with the feed containing the chemically flocculated cellular materials, debris and colloidal particulates; and
g) separating the flocculated bimolecular species of interest and the plurality of flocculated cellular material without the use of a centrifugation clarification step or a tangential flow microfiltration clarification step, wherein the level of total organic extractables measured in a feed filtered through the media after flushing is about 1-3 ppm.

The present invention is directed towards a process for reducing organic extractables from a primary clarification depth filter using an extraction system having a primary clarification depth filtration device containing a lower organic extractable media.

The present invention is directed towards primary clarification without the use of a primary clarification centrifugation step or primary clarification tangential flow microfiltration step. The depth filtration devices are able to filter high solids feeds containing particles having a particle size distribution of approximately 0.5 μm to 200 μm at a flow rate of about 10 litres/m²/hr to about 600 liters/m²/hr until the TMP reaches 20 psi. The primary clarification depth filter media taught herein include graded porous layers of varying pore ratings extracted with an organic solvent. The extraction solvent, HFE-72DE (Novec™ Engineered Fluid HFE-72DE by 3M™ St. Paul, Minn., USA) or one of its possible replacements (HFE-71DE, HCFC-141b, Vertrel MCA, or Vertrel MCA+), are all solvents for hydrocarbon and fluorocarbon greases and oils.

### BRIEF DESCRIPTION OF THE DRAWINGS

The accompanying drawings, which are incorporated in and constitute a part of this specification, illustrate the presently contemplated embodiments of the invention and, together with the description, serve to explain the principles of the invention.

FIGS. 1A. 1B, 1C, 1D, 1E and 1F depict different schematic embodiments of examples of primary clarification depth filters according to the invention, wherein FIGS. 1A, 1C and 1E depict primary clarification depth filters having at least 7 layers for use with polymer flocculant (smart polymer) treated feeds, and FIGS. 1B, 1D and 1F depict primary clarification depth filters having at least 8 layers for use with chemically treated feeds (acid treatment);

FIG. 2 depicts flushing curves for different embodiments of the primary clarification filters with non-extracted media at a working flow rate of 600 liters/m²/hr according to the invention;

FIG. 3 depicts flushing curves for multiple embodiments of the primary clarification filters with extracted media at a working flow rate of 600 liters/m²/hr according to the invention; and

FIG. 4 depicts flushing curves for multiple embodiments of the primary clarification filters with extracted media at a working flow rate of 100 liters/m²/hr according to the invention.

### DESCRIPTION OF THE EMBODIMENTS

All publications, patents and patent applications cited herein, whether supra or infra, are hereby incorporated by reference in their entirety to the same extent as if each individual publication, patent or patent application was specifically and individually indicated to be incorporated by reference.

For the purposes of this specification and appended claims, unless otherwise indicated, all numbers expressing quantities of ingredients, percentages or proportions of materials, reaction conditions, and other numerical values used in the specification and claims, are to be understood as being modified in all instances by the term “about” whether or not explicitly indicated. The term “about” generally refers to a range of numbers that one would consider equivalent to the recited value (i.e., having the same function or result). In many instances, the term “about” may include numbers that are rounded to the nearest significant figure.

Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass all sub ranges subsumed therein. For example, a range of “1 to 10” includes any and all sub ranges between (and including) the minimum value of 1 and the maximum value of 10, that is, any and all sub ranges having a minimum value of equal to or greater than 1 and a maximum value of equal to or less than 10, e.g., 5.5 to 10.

Before describing the present invention in further detail, a number of terms will be defined. Use of these terms does not limit the scope of the invention but only serve to facilitate the description of the invention.

As used herein, the singular forms “a,” “an,” and “the” include plural referents unless the context clearly dictates otherwise.

The term “biomolecule of interest”, as used herein, can be a desired target molecule such as, for example, a desired product or polypeptide of interest (e.g., an antibody), or it can be an undesirable entity, which needs to be removed from a sample containing the desired target molecule. Such undesirable entities include but are not limited to, for example, one or more impurities selected from host cell protein, DNA, RNA, protein aggregates, cell culture additives, viruses, endotoxins, whole cells and cellular debris. In addition, the biomolecule of interest may also be bound and precipitated by a stimulus responsive polymer or chemically treated (e.g., acid treatment) as described herein.

The term “capture step”, as used herein, generally refers to a method used for binding a target molecule with a stimulus responsive polymer or a chromatography resin, which results in a solid phase containing a precipitate of the target molecule and the polymer or resin. Typically, the target molecule is subsequently recovered using an elution step, which removes the target molecule from the solid phase, thereby resulting in the separation of the target molecule from one or more impurities. In various embodiments, the capture step can be conducted using a chromatography media, such as a resin, membrane or monolith, or a polymer, such as a stimulus responsive polymer, polyelectrolyte or polymer which binds the target molecule.

The term “cell culture additive” as used herein, refers to a molecule (e.g., a non-protein additive), which is added to a cell culture process in order to facilitate or improve the cell culture or fermentation process. In some embodiments according to the present invention, a stimulus responsive polymer, as described herein, binds and precipitates one or more cell culture additives. Exemplary cell culture additives include anti-foam agents, antibiotics, dyes and nutrients.

As used herein the phrase “cell culture” includes cells, cell debris and colloidal particles, biomolecule of interest, HCP, and DNA.

The term “chromatography”, as used herein, refers to any kind of technique which separates an analyte of interest (e.g. a target molecule) from other molecules present in a mixture. Usually, the analyte of interest is separated from other molecules as a result of differences in rates at which the individual molecules of the mixture migrate through a stationary medium under the influence of a moving phase, or in bind and elute processes.

The terms “chromatography resin” or “chromatography media”, are used interchangeably herein and refer to any kind of phase (e.g., a solid phase) which separates an analyte of interest (e.g., a target molecule) from other molecules present in a mixture. Usually, the analyte of interest is separated from other molecules as a result of differences in rates at which the individual molecules of the mixture migrate through a stationary solid phase under the influence of a moving phase, or in bind and elute processes. Examples of various types of chromatography media include, for example, cation exchange resins, affinity resins, anion exchange resins, anion exchange membranes, hydrophobic interaction resins and ion exchange monoliths.

The term “clarification step”, as used herein, generally refers to one or more steps used initially in the purification of biomolecules. The clarification step generally comprises removal of cells and/or cellular debris using one or more steps including any of the following alone or various combinations thereof. e.g. centrifugation and depth filtration, precipitation, flocculation and settling. In some embodiments, the present invention provides an improvement over the conventional and clarification step commonly used in various purification schemes. Clarification step generally involves the removal of one or more undesirable entities and is typically performed prior to a step involving capture of the desired target molecule. Another aspect of clarification is the removal of soluble and insoluble components in a sample which may later on result in the fouling of a sterile filter in a purification process, thereby making the overall purification process more economical.

In some embodiments, a purification process additionally employs one or more “chromatography steps”. Typically, these steps may be carried out, if necessary, after the separation of a target molecule from one or more undesired entities using a stimulus responsive polymer according to the present invention.

The terms “composition”, “solution”, or “sample” as used herein, refer to a mixture of a target molecule or a desired product to be purified using one or more stimulus responsive polymers or chemically treated (e.g. acid treatment) described herein along with one or more undesirable entities or impurities. In some embodiments, the sample comprises feedstock or cell culture media into which a target molecule or a desired product is secreted. In some embodiments, the sample comprises a target molecule (e.g., a therapeutic protein or an antibody) along with one or more impurities (e.g. host cell proteins, DNA, RNA, lipids, cell culture additives, cells and cellular debris). In some embodiments, the sample comprises a target molecule of interest which is secreted into the cell culture media.

The terms “Chinese hamster ovary cell protein” and “CHOP” as used interchangeably herein, refer to a mixture of host cell proteins (“HCP”) derived from a Chinese hamster ovary (“CHO”) cell culture. The HCP or CHOP is generally present as an impurity in a cell culture medium or lysate (e.g. a harvested cell culture fluid containing a protein or polypeptide of interest (e.g., an antibody or immunoadhesin expressed in a CHO cell). Generally, the amount of CHOP present in a mixture comprising a protein of interest provides a measure of the degree of purity for the protein of interest. Typically, the amount of CHOP in a protein mixture is expressed in parts per million relative to the amount of the protein of interest in the mixture.

The terms “contaminant”, “impurity”, and “debris”, are used interchangeably herein, refer to any foreign or objectionable material, including a biological macromolecule such as a DNA, an RNA, one or more host cell proteins (HCPs or CHOPs), endotoxins, viruses, lipids and one or more additives which may be present in a sample containing a protein or polypeptide of interest (e.g., an antibody) being separated from one or more of the foreign or objectionable molecules using a stimulus responsive polymer according to the present invention. In some embodiments, a stimulus responsive polymer described herein binds and precipitates a protein or polypeptide of interest from a sample containing the protein or polypeptide of interest and one or more impurities. In other embodiments, a stimulus responsive polymer described herein binds and precipitates one or more impurities, thereby to separate the polypeptide or protein of interest from one or more impurities.

It is understood that where the host cell is another mammalian cell type, an E. coli, a yeast cell, an insect cell, or a plant cell, HCP refers to the proteins, other than target proteins, found in a lysate of the host cell.

As used herein the term “depth filter” (e.g., gradient-density depth filter) achieves filtration within the depth of the filter material. A common class of such filters is those that comprise a random matrix of fibers bonded (or otherwise fixed), to form a complex, tortuous maze of flow channels. Particle separation in these filters generally results from entrapment by or adsorption to, the fiber matrix. The most frequently used depth filter media for bioprocessing of cell culture broths and other feedstocks consists of cellulose fibers, a filter aid such as DE, and a positively charged resin binder. Depth filter media, unlike absolute filters, retain particles throughout the porous media allowing for retention of particles both larger and smaller than the pore size. Particle retention is thought to involve both size exclusion and adsorption through hydrophobic, ionic and other interactions. The fouling mechanism may include pore blockage, cake formation and/or pore constriction. Depth filters are advantageous because they remove contaminants and also come in disposable formats thereby eliminating the validation issues.

As used herein the term “extractable(s)” refers to contaminants that in the presence of appropriate solvents can potentially migrate or be extracted from plastic and polymer compounds such as those materials used to make filter media or membranes, filter housing media or membrane support layer, an o-ring, or any other polymeric component of the filter, into a biopharmaceutical or pharmaceutical formulation and the like.

The term “extraction solvent”, as used herein, generally refers to a liquid substance with excellent cleaning properties. Their increased solvency, low surface tension, non-flammability and stability make it ideal for vapor degreasing applications. They are intended for medium to heavy-duty cleaning of soils such as oils, greases and waxes. Solvents include HFE-71DE, HFE-72DE, HCFC-141b, Vertrel MCA, or Vertrel MCA+) are all solvents for hydrocarbon and fluorocarbon greases and oils; the solvents also swell most elastomers. The high solvency and low toxicity make them an ideal replacement for ozone-depleting compounds, chlorinated solvents, and n-propyl bromide.

The term “flocculation” as used herein, refers to the addition of a flocculant, such as a polymer or chemically treated (e.g., acid treatment) described herein, to a solution in order to remove one or more suspended insoluble or soluble impurities. The polymer must be added to the solution at a concentration which allows for spontaneous formation of insoluble aggregates which can be removed from solution via typical solid-liquid separation methods.

The terms “isolating”, “purifying”, and “separating” are used interchangeably herein, in the context of purifying a target molecule (e.g., a polypeptide or protein of interest) from a composition or sample comprising the target molecule and one or more impurities, using a stimulus responsive polymer described herein. In some embodiments, the degree of purity of the target molecule in a sample is increased by removing (completely or partially) one or more impurities from the sample by using a stimulus responsive polymer, as described herein. In another embodiment, the degree of purity of the target molecule in a sample is increased by precipitating the target molecule away from one or more impurities in the sample.

As used herein the phrase “low or lower organic extractable media” refers to a media that when extracted with organic solvents results in the removal of extractables that can migrate from a material into a solvent including water under exaggerated conditions of time and temperature.

The term “monoclonal antibody” as used herein refers to an antibody obtained from a population of substantially homogeneous antibodies, i.e., the individual antibodies comprising the population are identical except for possible naturally occurring mutations that may be present in minor amounts.

The term “parts per million” or “ppm” are used interchangeably herein, and refer to a measure of purity of a desired target molecule (e.g. a target protein or antibody) purified using a stimulus responsive polymer described herein. Accordingly, this measure can be used either to gauge the amount of a target molecule present after the purification process or to gauge the amount of an undesired entity. In some embodiments, the units “ppm” are used herein to refer to the amount of an impurity in a solution, e.g., HCP or CHOP, in nanograms/milliliter of protein of interest in milligrams/milliliter (i.e., CHOP ppm=(CHOP ng/ml)/(protein of interest mg/ml). When the proteins are dried (e.g., by lyophilization), ppm refers to (CHOP ng)/(protein of interest mg)).

The terms “pI” or “isoelectric point” of a polypeptide, as used interchangeably herein, refer to the pH at which the polypeptide's positive charge balances its negative charge. pI can be calculated from the net charge of the amino acid residues or sialic acid residues of attached carbohydrates of the polypeptide or can be determined by isoelectric focusing.

The terms “precipitate”, precipitating” or “precipitation” as used herein, refer to the alteration of a bound (e.g., in a complex with a biomolecule of interest) or unbound polymer or other soluble species from an aqueous and/or soluble state to a non-aqueous and/or insoluble state.

As used herein the terms “pore size” and “nominal pore size” refer to the pore size which retains the majority of the particulate at 60-98% of the rated pore size.

As used herein interchangeably, the terms “polypeptide” or “protein”, generally refer to peptides and proteins having more than about ten amino acids. In some embodiments, a stimulus responsive polymer described herein is used to separate a protein or polypeptide from one or more undesirable entities present in a sample along with the protein or polypeptide. In some embodiments, the one or more entities are one or more impurities which may be present in a sample along with the protein or polypeptide being purified. As discussed, above, in some embodiments, a stimulus responsive polymer described herein specifically binds and precipitates a protein or polypeptide of interest upon the addition of a stimulus to the sample. In other embodiments, a stimulus responsive polymer described herein binds to and precipitates an entity other than the protein or polypeptide of interest such as, for example, host cell proteins, DNA, viruses, whole cells, cellular debris and cell culture additives, upon the addition of a stimulus.

As used herein the phrase “primary clarification depth filter” refers to a filter which is able to remove whole cells and cell debris thus accomplishing the primary clarification of a feed containing a target biomolecule of interest and a plurality of cellular debris and colloidal particulates without the use of a primary clarification centrifugation step or a primary clarification tangential flow microfiltration step.

The terms “protein of interest”, “target polypeptide”, “polypeptide of interest,” and “target protein,” are used interchangeably herein, and generally refer to a therapeutic protein or polypeptide, including but not limited to, an antibody purified using a stimulus responsive polymer according to the present invention.

In some embodiments, a “purification step” to isolate, separate or purify a polypeptide or protein of interest using a stimulus responsive polymer described herein, may be part of an overall purification process resulting in a “homogeneous” or “pure” composition or sample, which term is used herein to refer to a composition or sample comprising less than 100 ppm HCP in a composition comprising the protein of interest, alternatively less than 90 ppm, less than 80 ppm, less than 70 ppm, less than 60 ppm, less than 50 ppm, less than 40 ppm, less than 30 ppm, less than 20 ppm, less than 10 ppm, less than 5 ppm, or less than 3 ppm of HCP. As used herein “primary clarification” includes the removal of aggregated cellular biomass, including flocculated cellular debris and colloidal particulates with a size larger than about 10 microns (μm) or smaller particles with the use of a flocculating agent.

The term “salt”, as used herein, refers to a compound formed by the interaction of an acid and a base. Various salts which may be used in various buffers employed in the methods described herein include, but are not limited to, acetate (e.g. sodium acetate), citrate (e.g., sodium citrate), chloride (e.g., sodium chloride), sulphate (e.g., sodium sulphate), or a potassium salt. The term “solvent”, as used herein, generally refers to a liquid substance capable of dissolving or dispersing one or more other substances to provide a solution. Solvents include aqueous and organic solvents, where useful organic solvents include a non-polar solvent, ethanol, methanol, isopropanol, acetonitrile, hexylene glycol, propylene glycol, and 2,2-thiodiglycol.

The terms “target molecule”, “target biomolecule”, “desired target molecule” and “desired target biomolecule,” are used interchangeably herein, and generally refer to a polypeptide or product of interest, which is desired to be purified or separated from one or more undesirable entities, e.g., one or more impurities, which may be present in a sample containing the polypeptide or product of interest.

As used herein the term “throughput” means the volume filtered through a filter.

In the present invention, the use of open graded layers allows the larger particles to penetrate and become captured within the depth of the filters, rather than collecting on the surface.

The advantage is higher throughput, and retention of large solids (about 0.5 microns to about 200 microns) while eliminating the problem of cake formation. The use of open pores in the primary clarification filters provides these depth filters with the linear increase in pressure with the solid retention with no significant increase in the pressure and hence resulting in high throughputs. The structural dimension of the filter in combination with the optimization of layers (pore sizes and thickness) gives exceptional filtration properties which can retain high amount of solids.

In the present invention, the use of open graded layers allows the larger flocculated particles in the feed stream to penetrate into the depth of the filter, and become captured within the pores of the filter rather than collect on the surface. The primary clarification depth filter provided herein is arranged such that the “open” top layer(s) constitute the prefiltration zone of the depth filters in order to capture larger flocculated particles, while the bottom layer(s) constitute the polishing zone which captures the smaller residual aggregated flocculated particles.

The primary clarification depth filter having this type of arrangement is exhibits advantages such as (i) higher throughput, (ii) the retention of larger flocculated solids; and (iii) the elimination of the problem of cake formation. The use of such open pores in the primary clarification filter taught herein provides a linear increase in pressure with the solids retention, with no significant increase in the pressure, resulting in higher, more desirable throughputs.

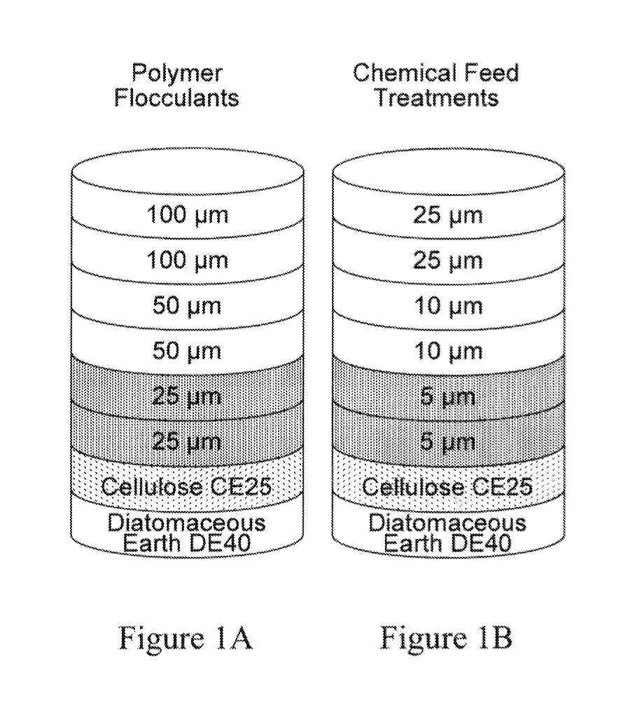

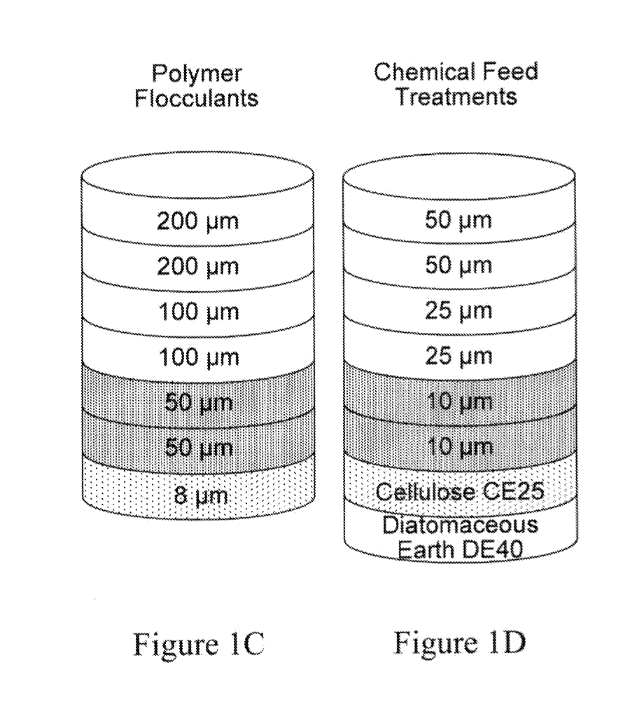

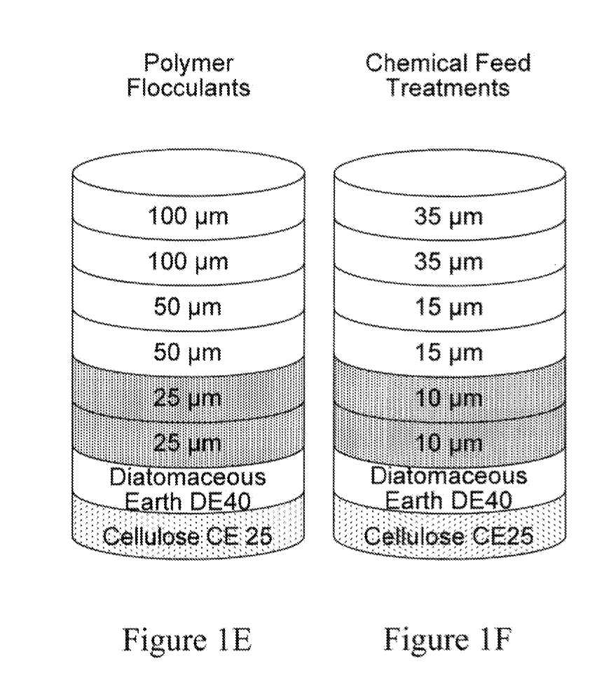

Examples of primary clarification depth filters according to the invention are depicted in FIGS. 1A, 1B, 1C, 1D, 1E and 1F, wherein FIGS. 1A, 1C and 1E depict primary clarification depth filters having at least 7 or 8 layers, and are used when the cell-culture feeds are treated with a polymer flocculant (e.g., smart polymer or traditional flocculant).

FIGS. 1B, 1D and 1F depict primary clarification depth filters having at least 7 layers, and are used when the cell-culture feeds are treated with a chemically treated feeds (e.g., acid treatment).

The primary clarification depth filter depicted in FIG. 1A shows a primary clarification depth filter used when the cell-culture feeds are treated with a polymer flocculant (e.g., smart polymer) having two (upper) layers with a nominal pore size of about 100 microns of a non woven such as polypropylene about 0.4 cm thick, having two more layers with a nominal pore size of about 50 microns of a non woven such as polypropylene about 0.4 cm thick, having two additional layers with a nominal pore size of about 25 microns of a non woven such as polypropylene about 0.4 cm thick, followed by a single layer about 0.35 cm thick of a material such as cellulose (CE25) for example, and another single layer about 0.35 cm thick of a material such as diatomaceous earth (DE40) for example.

The primary clarification depth filter depicted in FIG. 1B shows a primary clarification depth filter used when the cell-culture feeds are chemically treated (e.g., acid treatment) having two (upper) layers with a nominal pore size of about 25 microns of a non woven such as polypropylene about 0.4 cm thick, having two more layers with a nominal pore size of about 10 microns of a non woven such as polypropylene about 0.4 cm thick, having two additional layers with a nominal pore size of about 5 microns of a non woven such as polypropylene about 0.4 cm thick, followed by a single layer about 0.35 cm thick of a material such as cellulose (CE25) for example, and followed by another single of layer about 0.35 cm thick of a material such as diatomaceous earth (DE40) for example. Either the cellulose or diatomaceous earth layer can be selected as the lowest (bottom) layer.

The primary clarification depth filter depicted in FIG. 1C shows a primary clarification depth filter used when the cell-culture feeds are treated with a polymer flocculant (e.g., smart polymer) having two (upper) layers with a nominal pore size of about 100 microns comprising a non woven such as polypropylene about 0.4 cm thick, having two more layers with a nominal pore size of about 100 microns of a non woven such as polypropylene about 0.4 cm thick, having two additional layers with a nominal pore size of about 100 microns comprising a non woven such as polypropylene about 0.4 cm thick, followed by a single layer (bottom) about 8 microns thick of a non woven such as polypropylene about 0.2 cm thick.

The primary clarification depth filter depicted in FIG. 11) shows a primary clarification depth filter used when the cell-culture feeds are chemically treated (e.g. acid treatment) having two (upper) layers with a nominal pore size of about 50 microns comprising a non woven such as polypropylene about 0.4 cm thick, having two additional layers with a nominal pore size of about 25 microns of a non woven such as polypropylene about 0.4 cm thick, having two more layers with a nominal pore size of about 10 microns of a non woven such as polypropylene about 0.4 cm thick, followed by a single layer about 0.35 cm thick of a material such as cellulose (CE25) for example, and followed by another single of layer about 0.35 cm thick of a material such as diatomaceous earth (DE40) for example. Either the cellulose or diatomaceous earth layer can be selected as the lowest (bottom) layer.

The primary clarification depth filter depicted in FIG. 1E shows a primary clarification depth filter used when the cell-culture feeds are treated with a polymer flocculant (e.g., smart polymer) having two (upper) layers with a nominal pore size of about 100 microns comprising a non woven such as polypropylene about 0.4 cm thick, having two more layers with a nominal pore size of about 50 microns of a non woven such as polypropylene about 0.4 cm thick, having two additional layers with a nominal pore size of about 25 microns comprising a non woven such as polypropylene about 0.4 cm thick, followed by a layer about 0.35 cm thick of a material such as cellulose (CE25) for example, and followed by another single of layer about 0.35 cm thick of a material such as diatomaceous earth (DE40) for example.

The primary clarification depth filter depicted in FIG. 1F shows a primary clarification depth filter used when the cell-culture feeds are chemically treated (e.g., acid treatment) having two (upper) layers with a nominal pore size of about 35 microns comprising a non woven such as polypropylene about 0.4 cm thick, having two more layers with a nominal pore size of about 15 microns of a non woven such as polypropylene about 0.4 cm thick, having two additional layers with a nominal pore size of about 10 microns comprising a non woven such as polypropylene about 0.4 cm thick, followed by a single layer about 0.35 cm thick of a material such as cellulose (CE25) for example, and followed by another single of layer 0.35 cm thick of a material such as diatomaceous earth (DE40) for example. Either the cellulose or diatomaceous earth layer can be selected as the lowest (bottom) layer.

The efficiency parameter K, is used herein to describe the filter efficiency while normalizing for the solid content of a particularly feedstock. The parameter K allows for filtration of feeds with different solids content to be effectively compared.

The following examples are provided so as to provide those of ordinary skill in the art with a complete disclosure and description of how to make the compositions of the invention and how to practice the methods of the invention and are not intended to limit the scope of what the inventor regards as his invention. Efforts have been made to insure accuracy with respect to numbers used (e.g. amounts, temperature, etc.), but some experimental errors and deviation should be accounted for. Unless indicated otherwise, temperature is in degrees C., chemical reactions were performed at atmospheric pressure or transmembrane pressure, as indicated, the term “ambient temperature” refers to approximately 25° C. and “ambient pressure” refers to atmospheric pressure. The invention will be further clarified by the following examples which are intended to be exemplary of the invention.

### EXAMPLES

### Example 1

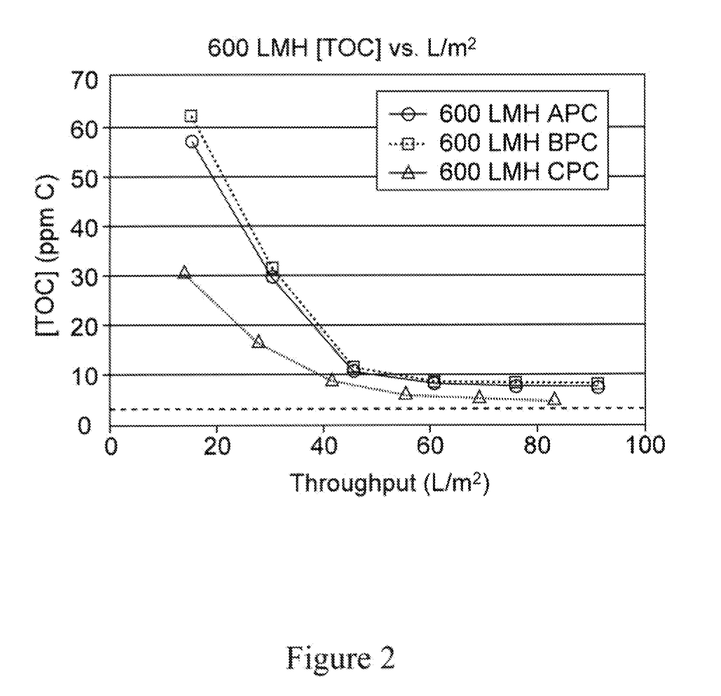

FIG. 2 depicts flushing curves for primary clarification filters with non-extracted media at a working flow rate of 600 liters/m²/hr.

In a representative experiment, depth filter comprising of graded layers of non-woven fibers, cellulose, and diamatoceous earth (DE) or non-woven fibers was flushed for approximately 100 L/m² at a flow rate of 600 liters/m²/hr. The flushing curves for the depth filter comprising of graded layers of non-woven fibers, cellulose, and diamatoceous earth (APC and BPC) have a TOC of approximately 8-10 ppm whereas depth filter comprising of graded layers of non-woven fibers (CPC) has a TOC of approximately 4 ppm as shown in FIG. 1. The TOC (ppm) of the control depth filter (D0HC) is between 1-3 ppm for a flush volume of approximately 100 L/m².

### Example 2

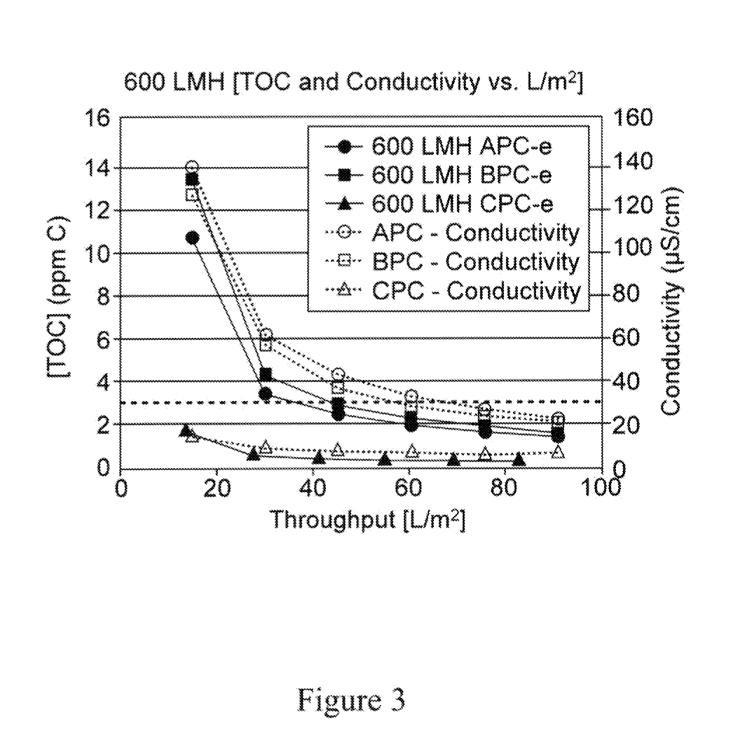

FIG. 3 depicts flushing curves for multiple embodiments of the primary clarification filters with extracted media at a working flow rate of 600 liters/m²/hr according to the invention.

In a representative experiment, depth filters of graded layers of extracted non-woven fibers, cellulose, and diamatoceous earth or extracted non-woven fibers were flushed for approximately 100 L/m² at a flow rate of 600 liters/m²/hr. The rolls of non-woven filter media (12.5″ in diameter and 16″ in width) are extracted with hydrofluorocarbon solvent (HFE-72E) from 3M in the TSC extractor for a spraying time of 1200 min and drying time of 1500 min. The flushing curves for the depth filter comprising of graded layers of non-woven fibers, cellulose, and diamatoceous earth (APC and BPC) have a TOC of approximately 1-3 ppm for a flush volume of approximately 100 L/m² whereas depth filter comprising of graded layers of non-woven fibers (CPC) has a TOC of lesser than 1 ppm for no flush volume. The TOC (ppm) of the control depth filter (D0HC) is between 1-3 ppm for a flush volume of approximately 100 L/m². Even though APC and BPC depth filters with extracted non-woven media have roughly the same flush volume of 100 L/m² as D0HC to reach the target the TOC of 1-3 ppm; the column volume of APC and BPC is double than D0HC which suggests that the flushing volume is reduced by half which can significantly reduce the flushing for the overall process with the higher throughput of the primary clarification depth filters.

### Example 3

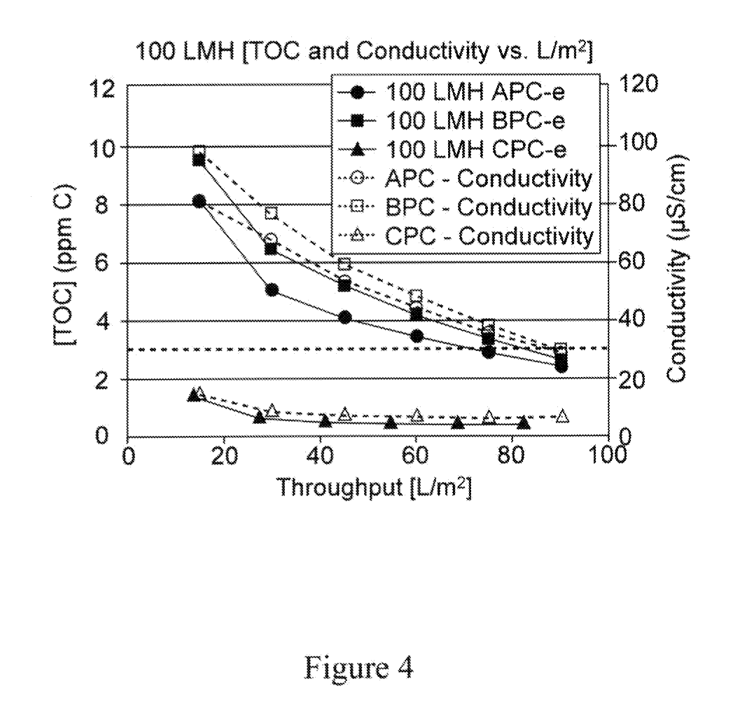

FIG. 4 depicts flushing depicts curves for multiple embodiments of the primary clarification filters with extracted media at a working flow rate of 100 liters/m²/hr according to the invention.

In a representative experiment, depth filter comprising of graded layers of extracted non-woven fibers, cellulose, and diamatoceous earth or extracted non-woven fibers was flushed for approximately 100 L/m² at a flow rate of 600 liters/m²/hr. The rolls of non-woven filter media (12.5″ in diameter and 16″ in width) are extracted with hydrofluorocarbon solvent (HFE-72E) from 3M in the TSC extractor for a spraying time of 1200 min and drying time of 1500 min. The flushing curves for the depth filter comprising of graded layers of non-woven fibers, cellulose, and diamatoceous earth (APC and BPC) have a TOC of approximately 1-3 ppm for a flush volume of approximately 90 L/m² whereas depth filter comprising of graded layers of non-woven fibers (CPC) has a TOC of lesser than 1 ppm for no flush volume. The desired levels of TOC (ppm) of the depth filters are between 1-3 ppm for a flush volume of approximately 100 L/m².

### Example 4

### Static Soaking Experiment for the Extracted and Non-Extracted Non-Woven Fibers

In a representative experiment, disks of non-woven filter media are extracted with hydrofluorocarbon solvent (Vertrel MCA+ from Dupont and HFE-72E from 3M) for a soaking time of 1 min and drying time of 1 hour at 80° C. The extracted and non-extracated non-woven disks of 23 cm² were soaked in 50 ml Milli-Q water for 1 hour and analyzed for total organic extractables (TOC). The TOC was lesser than 1 ppm for all the extracted samples, however the TOC's was higher for all the non-woven extracted samples. Table I compares the total organic extractables (TOC) of extracted and non extracted non woven fibers.

TABLE 1
Comparison of the total organic extractables
(TOC) for extracted and non-extracted media.

| Material of | Nominal Pore | Extraction | TOC |
|---|---|---|---|
| Construction | Ratings (μm) | Solvent | (ppm) |
| Polypropylene | 200 | None | 3 |
| Polypropylene | 200 | Vertrel MCA+ | <1 |
| Polypropylene | 200 | HFE-72E | <1 |
| Polypropylene | 50 | None | 5 |
| Polypropylene | 50 | Vertrel MCA+ | <1 |
| Polypropylene | 50 | HFE-72E | <1 |
| Polypropylene | 5 | None | 4 |
| Polypropylene | 5 | Vertrel MCA+ | <1 |
| Polypropylene | 5 | HFE-72E | <1 |

### Example 5

### Filtration Performance of Depth Filters Removal of Aggregated and Small Biomolecule Particulates with Extracted and Non-Extracted Media

APC filter devices from Examples 1-2 were tested for filtration performance using the following method. The unclarified cell culture harvest was treated with 1M glacial acetic acid to adjust the pH to 4.8 and stirred for 30 minutes. Depth filters were run with untreated and acid treated unclarified feed after flushing out with the Milli-Q water with the TMP across each filter monitored by pressure transducers. The depth filters were first flushed with ≧about 50 L of Milli-Q water for each square meter of filter area at 600 L/m²/h to wet the filter media and flush out extractables. Untreated and acid precipitated unclarified harvest was loaded at 100 L/m²/h until the TMP across any one filter reached 20 psig.

Table 2 compares the filter throughput of Millistak® filter (D01-HC) with extracted and non-extracted primary clarification depth filter for the acid treated feed.

TABLE 2
Comparison of the Primary Clarification (APC) Depth Filter for the filtration throughput
for acid treated feed (pH = 4.8) with extracted and non-extracted media.

| | | Filter | | PCV | Turbidity | TP | TP | K |
|---|---|---|---|---|---|---|---|---|
| Feed | Treatment | Type | pH | (%) | (NTU) | (L/m²) | (V_{f}/Vₘ) | (%) |
| CHO-S | Untreated | D0HC | 6.9 | 3.8 | 90 | 100 | 11 | 42 |
| CHO-S | Acid | APC (Non- | 4.8 | 3.9 | 1 | 245 | 18 | 70 |
| | treated | extracted) | | | | | | |
| CHO-S | Acid | APC | 4.8 | 3.9 | 3 | 250 | 18 | 70 |
| | treated | (Extracted) | | | | | | |

### Example 6

### Filtration Performance of Depth Filters Removal of Aggregated and Small Biomolecule Particulates with Extracted and Non-Extracted Media

CPC filter devices from Examples 1-2 were tested for filtration performance using the following method. The depth filters were run with untreated and SmP treated unclarified feed after flushing out with the Milli-Q water with the TMP across each filter monitored by pressure transducers. The unclarified cell culture harvest was treated with 0.2 wt % smart polymer (SmP) dose (wt %) and stirred for 15 minutes. The depth filters were first flushed with ≧about 50 L of Milli-Q water for each square meter of filter area at 600 L/m²/h to wet the filter media and flush out extractables. Untreated and SmP treated unclarified harvest were loaded at 100 L/m²/h until the TMP across any one filter reached 20 psig.

Table 3 compares the filter throughput of Millistak® filters (D0HC) with extracted and non-extracted Primary clarification depth filter for the filtration of the feed described in Example 3. (0.2% (w/v) smart polymer (SmP) treated feed).

TABLE 3
Comparison of the Primary Clarification (CPC) Depth Filter described in Example
1-2 for the filtration throughput of SMP treated feed with 0.2% (w/v).

| | | Filter | Dose | PCV | Turbidity | TP | TP | K |
|---|---|---|---|---|---|---|---|---|
| Feed | Treatment | Type | (%) (w/v) | (%) | (NTU) | (L/m²) | (V_{f}/Vₘ) | (%) |
| CHO-S | Untreated | D0HC | NA | 3.8 | 90 | 100 | 11 | 42 |
| CHO-S | SMP | CPC (Non- | 0.2 | 4.0 | 2 | 260 | 19 | 75 |
| | treated | extracted) | | | | | | |
| CHO-S | SMP | CPC | 0.2 | 4.0 | 2 | 275 | 20 | 80 |
| | treated | (Extracted) | | | | | | |

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in applications claiming priority from this or a related application. Such claims, whether directed to a different invention or to the same invention, and whether broader, narrower, equal, or different in scope to the original claims, also are regarded as included within the subject matter of the inventions of the present disclosure.

What is claimed is:

1. A process for reducing organic extractables released from a primary clarification depth filtration media such that the level of total organic extractables measured in a feed filtered through the media after flushing is about 1-3 ppm, the process comprising:
a) providing a depth filtration device having a porous depth filter media;
b) extracting from the media with organic solvents; and
c) flushing the media at flow rates ranging from about 10 litres/m²/hr to about 600 litres/m²/hr such that the level of total organic extractables measured in a feed stock filtered through the media after flushing is about 1-3 ppm.

2. The process of claim 1, wherein the depth filter comprises at least 2 graded layers of non-woven fibers.

3. The process of claim 1, wherein the extraction solvent is selected from the consisting of HFE-72DE, HFE-71DE, HCFC-141b, Vertrel MCA, or Vertrel MCA+, all solvents for hydrocarbon and fluorocarbon greases and oils.

4. The process of claim 1, wherein the depth filter comprises at least 3 graded layers of non-woven fibers.

5. The process of claim 4, wherein the graded layers have a total thickness of about 0.3 cm to about 3 cm.

6. The process of claim 1, wherein the cellular debris and the colloidal particulates have a particle size distribution from about 0.5 μm to about 200 μm, and a mean particle size greater than about 10 μm.

7. The process of claim 2, wherein the depth filter media comprises a composite of graded layers of non-woven fibers, cellulose, and diamatoceous earth having an open nominal pore size rating sufficient to filter the chemically flocculated feedstock.

8. The process of claim 1, wherein the target biomolecule of interest includes monoclonal antibodies (mAbs), polyclonal antibodies, and biotherapeutics.

9. The process of claim 1, wherein the chemical flocculant is a polymer or an acid.

10. The process of claim 1, wherein the chemical flocculant is a smart polymer.

11. The process of claim 10, wherein the smart polymer is a modified polyamine.

12. The process of claim 9, wherein the acid is acetic acid.

13. The process of claim 4, wherein the non-woven fibers comprise polypropylene, polyethylene, polyester, or nylon.

14. A primary clarification depth filtration process using a primary clarification depth filtration device containing a porous media having a lower level of organic extractables released from the media after flushing, the process comprising:
a) providing a primary clarification depth filtration device having a porous depth filter media;
b) extracting from the media with organic solvents;
c) flushing the media at flow rates ranging from about 10 litres/m²/hr to about 600 litres/m²/hr;
d) resulting in a lower organic extractable media such that the level of total organic extractables measured in a feed stock filtered through the media is about 1-3 ppm; and
e) running a feed stock through the media.

15. The process of claim 14, wherein the depth filter comprises at least 2 graded layers of non-woven fibers.

16. The process of claim 14, wherein the extraction solvent is selected from the consisting of HFE-72DE, HFE-71DE, HCFC-141b, Vertrel MCA, or Vertrel MCA+, all solvents for hydrocarbon and fluorocarbon greases and oils.

17. The process of claim 14, wherein the depth filter comprises at least 3 graded layers of non-woven fibers.

18. The process of claim 17, wherein the graded layers have a total thickness of about 0.3 cm to about 3 cm.

19. The process of claim 14, wherein the cellular debris and the colloidal particulates have a particle size distribution from about 0.5 μm to about 200 μm, and a mean particle size greater than about 10 μm.

20. The process of claim 15, wherein the depth filter media comprises a composite of graded layers of non-woven fibers, cellulose, and diamatoceous earth having an open nominal pore size rating sufficient to filter the chemically flocculated feedstock.

21. The process of claim 14, wherein the target biomolecule of interest includes monoclonal antibodies (mAbs), polyclonal antibodies, and biotherapeutics.

22. The process of claim 14, wherein the chemical flocculant is a polymer or an acid.

23. The process of claim 14, wherein the chemical flocculant is a smart polymer.

24. The process of claim 23, wherein the smart polymer is a modified polyamine.

25. The process of claim 22, wherein the acid is acetic acid.

26. The process of claim 14, wherein the non-woven fibers comprise polypropylene, polyethylene, polyester, or nylon.
